(19) United States
(12) Patent Application Publication
(10) Pub. No.: US 2015/0265348 A1
(43) Pub. Date: Sep. 24, 2015

(54) ELECTROPHYSIOLOGY SYSTEM

(71) Applicant: BOSTON SCIENTIFIC SCIMED, INC., MAPLE GROVE, MN (US)

(72) Inventors: BOAZ AVITALL, WHITEFISH BAY, WI (US); JOSEF V. KOBLISH, SUNNYVALE, CA (US); MICHAL WEISMAN, WINCHESTER, MA (US); RAJ SUBRAMANIAM, FREMONT, CA (US)

(73) Assignee: BOSTON SCIENTIFIC SCIMED, INC., MAPLE GROVE, MN (US)

(21) Appl. No.: 14/661,852

(22) Filed: Mar. 18, 2015

Related U.S. Application Data
(60) Provisional application No. 61/955,087, filed on Mar. 18, 2014.

(51) Int. Cl. A61B 18/18; A61B 5/0452; A61B 5/044; A61B 5/042

(52) U.S. Cl. CPC A61B 18/18; A61B 5/0422; A61B 5/0452; A61B 5/044

(57) ABSTRACT

A radio frequency (RF) ablation system and methods for using the radio frequency ablation system are disclosed. The RF ablation system may include an elongated member, an RF generator, and a processor. The elongated member may include a distal portion including one or more electrodes and the RF generator may be operatively coupled to one or more of the electrodes. The processor, which may be coupled to one or more of the electrodes, may obtain an output signal from the electrodes and may monitor changes in a frequency spectra of an electrical signal received with the output signal. The processor may determine a level of one or more characteristics that are proportional to an amplitude of the electrical signal, proportional to the frequency of the electrical signal, or proportional to both of the amplitude and the frequency of the electrical signal.

### CROSS-REFERENCE TO RELATED APPLICATIONS

This application claims priority under 35 U.S.C. §119 to U.S. Provisional Application Ser. No. 61/955,087, filed Mar. 18, 2014, the entirety of which is incorporated herein by reference.

### TECHNICAL FIELD

The disclosure is directed to systems and methods for providing therapies. More particularly, the disclosure is directed to systems and methods for mapping and ablating tissue.

### BACKGROUND

Ablation is the removal of material from the surface of any object, for example, including, but not limited to, the deterioration and/or modulation of a material at or near a surface of any object. Illustratively, cardiac ablation may work by scarring, destroying and/or otherwise modulating tissue of a heart to correct and/or modify abnormal electrical signals traveling through the heart.

In a biological setting, aberrant conductive pathways disrupt the normal path of the heart's electrical impulses. For example, conduction blocks can cause the electrical impulses to degenerate into several circular wavelets that disrupt the normal activation of the atria or ventricles. The aberrant conductive pathways create abnormal, irregular, and sometimes life-threatening heart rhythms called arrhythmias. Ablation is one way of treating arrhythmias and restoring normal contraction through tissue modulation. The sources of the aberrant pathways (called focal arrhythmia substrates) are located or mapped using mapping electrodes situated in a desired location. After mapping, the physician may ablate the aberrant tissue. In radio frequency (RF) ablation, RF energy is directed from the ablation electrode through tissue to an electrode to ablate the tissue and form a lesion.

### SUMMARY

The present disclosure relates generally to systems and methods for providing therapies and for performing analyses while providing the therapies. It is contemplated that the analyses include determining levels of contact force between an ablation catheter and a target area and/or lesion maturation on a target area.

Accordingly, in one illustrative instance, a system may include an elongated member, a radio frequency (RF) generator, and a processor. The elongated member may have a distal portion that includes one or more electrodes. The radio frequency generator may be operatively coupled to one or more of the electrodes to generate energy that may be conveyed to one of the one or more electrodes. The processor may be operatively coupled to one or more of the electrodes and may be capable of obtaining output signals from one or more of the electrodes, where one or more of the output signals include an electrogram (EGM) reading. In some instances, the processor may be capable of monitoring changes in frequency spectra of the EGM in a frequency domain

In another illustrative instance, a method may include positioning a distal portion of an elongated member at a location proximate a target area and obtaining output signals from each bipolar microelectrode pair of the positioned distal portion of the elongated member, where one or more of the output signals comprises an electrical reading. In the method, frequency spectra of the electrical readings of the output signals in the frequency domain may be monitored over time. In some cases, the positioned distal portion of the elongated member may include an electrode capable of applying RF energy to the target area and a plurality of microelectrodes distributed about the electrode and electrically isolated therefrom, where the plurality of microelectrodes may define the bipolar microelectrode pairs positioned proximate the target area.

In another illustrative instance, a system may include an elongated member, a radio frequency generator, a processor, and an indicator. The elongated member may have a distal portion that includes one or more RF energy electrodes capable of applying RF energy to the target tissue and a plurality of microelectrodes distributed about the one or more RF energy electrodes and electrically isolated therefrom, where the plurality of microelectrodes define a plurality of bipolar microelectrode pairs and each bipolar microelectrode pair is capable of generating an output signal. The RF generator may be operatively coupled to one or more of the electrodes to provide energy to be conveyed to one or more of the electrodes. The processor may be operatively coupled to one or more of the electrodes and while applying RF energy to the target tissue, the processor may be capable of obtaining output signals from each of the bipolar microelectrode pairs, where one or more of the output signals include an EGM reading. Frequency spectra of the EGMs may be monitored by the processor while the catheter is being positioned or is being used in an RF energy application procedure. The indicator may be in communication with the processor and may indicate a level of lesion maturation that is proportional to changes in the frequency spectra of the EGM during the ablation procedure.

The above summary is not intended to describe each embodiment or every implementation of the present disclosure. Advantages and attainments, together with a more complete understanding of the disclosure, will become apparent and will be appreciated by referring to the following description and claims taken in conjunction with the accompanying drawings.

### BRIEF DESCRIPTION OF THE DRAWINGS

The aspects of the disclosure may be more completely understood in consideration of the following detailed description of various embodiments in connection with the accompanying drawings, in which:

FIG. 1 is a schematic illustration of a radio frequency (RF) ablation system that may be used in accordance with various examples of the present disclosure;

FIG. 2 is a schematic illustration showing a conventional ablation catheter on the left and an embodiment of an RF ablation catheter of the present disclosure on the right;

FIG. 3 is a schematic flow diagram of a method of using an RF ablation system that may be used in accordance with various examples of the present disclosure;

FIG. 4 is a schematic illustration of electrical signals obtained from electrodes of an RF ablation system that may be used in accordance with various examples of the present disclosure;

FIG. 5 is a schematic illustration of electrical signals obtained from electrodes of an RF ablation system that may be used in accordance with various examples of the present disclosure;

FIG. 6 is a schematic illustration of electrical signals obtained from electrodes of an RF ablation system in the time domain on the left and in the frequency domain on the right, which may be used in accordance with various examples of the present disclosure;

FIGS. 7-9 are schematic illustrations of electrical signals obtained from electrodes of an RF ablation system in the time domain and the frequency domain over a portion of an ablation process, which may be used in accordance with various examples of the present disclosure.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Any relative terms, such as first, second, third, right, left, bottom, top, high, low, etc., used herein in connection with a feature are just that and are not meant to be limiting other than to be indicative of the relative relationship of the modified feature with respect to another feature.

Although some suitable dimensions, ranges and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative instances and are not intended to limit the scope of the disclosure. Selected features of any illustrative instance may be incorporated into an additional instance unless clearly stated to the contrary.

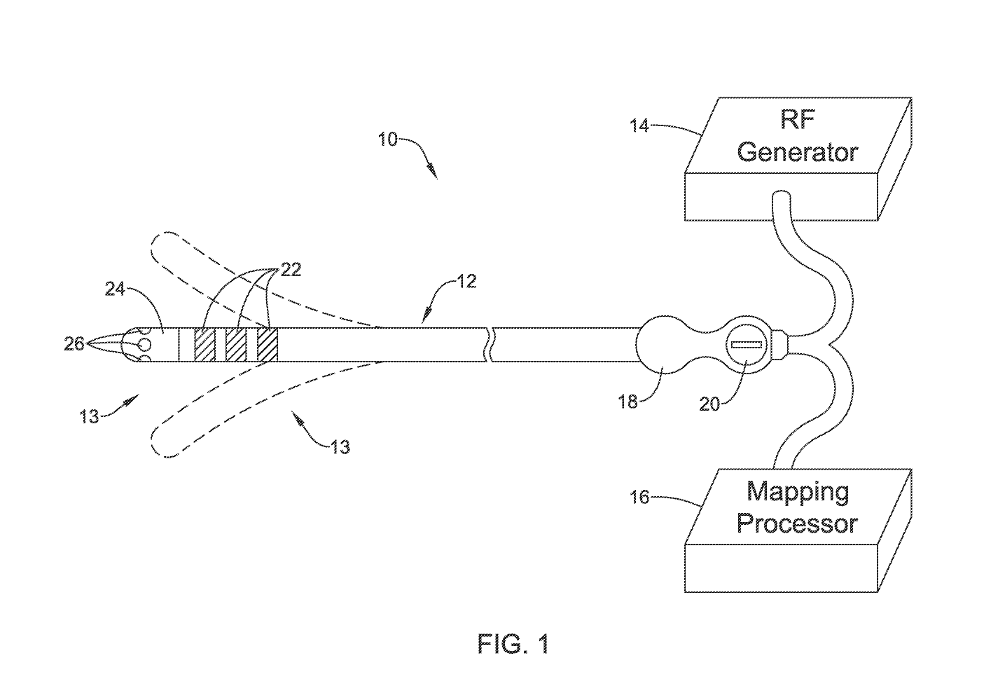

FIG. 1 is an illustrative radio frequency (RF) ablation system 10. As shown in FIG. 1, the system 10 may include one or more of an elongated member 12 (e.g., an elongated member 12, such as an ablation and/or mapping catheter, or other elongated member), an RF generator 14, and a processor 16 (e.g., a mapping processor, ablation processor, and/or other processor). Illustratively, the elongated member 12 may be operatively coupled to at least one or more (e.g., one or both) of the RF generator 14 and the processor 16. Alternatively, or in addition, a device, other than the elongated member 12, that may be utilized to apply RF energy to and/or map a target area may be operatively coupled to at least one or more of the RF generator 14 and the processor 16. The RF ablation system 10 may include any of one or more other features, as desired. In one instance, the RF ablation system 10 may include noise artifact isolators (not shown), where the microelectrodes 26 may be electrically insulated from the exterior wall by the noise artifact isolators.

In one illustrative example, the RF generator 14 may be coupled to one or more electrodes of the elongated member 12. The coupling between the RF generator 14 and one or more electrodes may be capable of facilitating the conveyance of RF energy generated by the RF generator 14 to the coupled electrodes.

In some instances, the elongated member 12 may include a handle 18, which may have an actuator 20 (e.g., a control knob or other actuator). The handle 18 (e.g., a proximal handle) may be positioned at a proximal end of the elongated member 12, for example. Illustratively, the elongated member 12 may include a flexible body having a distal portion which may include the one or more electrodes. For example, the distal portion of the elongated member 12 may include one or more of a plurality of ring electrodes 22, tissue ablation electrodes 24, and/or microelectrodes 26 (e.g., mapping microelectrodes, which may be referred to as pin electrodes, or other microelectrodes) disposed or otherwise positioned within and/or electrically isolated from the tissue ablation electrode 24.

In some instances, the ablation system 10 may be utilized in ablation (e.g., RF energy application) procedures on a patient. Illustratively, in such procedures the elongated member 12 may be configured to be introduced into or through vasculature of a patient and/or into or through any other lumen or cavity. In one example, the elongated member 12 may be inserted through the vasculature of the patient and into one or more chambers of the patient's heart (e.g., a target area). When in the patient's vasculature or heart, the elongated member 12 may be used to map and/or ablate myocardial tissue using the ring electrodes 22, the microelectrodes 26 and/or the tissue ablation electrode 24. In some instances, the tissue ablation electrode 24 may be configured to apply RF energy to myocardial tissue of the heart of a patient.

The elongated member 12 may be steerable to facilitate navigating the vasculature of a patient and/or navigating other lumens and/or cavities of the patient. Illustratively, a distal portion 13 of the elongated member 12 may be deflected by manipulation of the actuator 20 to effect steering the elongated member 12. In some instances, the distal portion 13 of the elongated member 12 may be deflected to position the tissue ablation electrodes 24 and/or the microelectrodes 26 adjacent target tissue or to position the distal portion 13 of the elongated member 12 for any other purpose. Additionally, or alternatively, the distal portion 13 of the elongated member 12 may have a pre-formed shape adapted to facilitate positioning the tissue ablation electrode 24 and/or the microelectrodes 26 adjacent a target tissue. Illustratively, the preformed shape of the distal portion 13 of the elongated member 12 may be a radiused shape (e.g., a generally circular shape or a generally semi-circular shape) and/or may be oriented in a plane transverse to a general longitudinal direction of the elongated member 12.

In some instances, the microelectrodes 26 of the elongated member 12 may be circumferentially distributed about the tissue ablation electrode 24 and/or electrically isolated therefrom. The microelectrodes 26 may be capable of operating, or configured to operate, in unipolar or bipolar sensing modes. In some cases, the plurality of microelectrodes 26 may define and/or at least partially form one or more bipolar microelectrode pairs 28.

In an illustrative instance, the elongated member 12 may have three microelectrodes 26 (e.g., a first microelectrode 26a, a second microelectrode 26b, and a third microelectrode 26c) distributed about the circumference of the tissue ablation electrode 24, such that the circumferentially spaced microelectrodes may form respective bipolar microelectrode pairs 28 of adjacent microelectrodes 26 (e.g., a first bipolar microelectrode pair 28a may include the first microelectrode 26a and the second microelectrode 26b, a second bipolar microelectrode pair 28b may include the second microelectrode 26b and the third microelectrode 26c, and a third bipolar microelectrode pair 28c may include the third microelectrode 26c and the first microelectrode 26a). Each bipolar microelectrode pair 28 may be capable of generating, or may be configured to generate, an output signal corresponding to a sensed electrical activity (e.g., an electrogram (EGM) reading) of the myocardial tissue proximate thereto.

Additionally or alternatively to the circumferentially spaced microelectrodes 26, the elongated member 12 may include one or more forward facing microelectrodes 26 (not shown). The forward facing microelectrodes 26 may be generally centrally located within the tissue ablation electrode 24 and/or positioned at an end of a tip of the elongated member 12.

In some examples, the microelectrodes 26 may be operatively coupled to the processor 16 and the generated output signals from the microelectrodes 26 may be sent to the processor 16 of the ablation system 10 for processing in one or more manners discussed herein and/or for processing in other manners. Illustratively, an EGM reading or signal of an output signal from a bipolar microelectrode pair 28 may at least partially form the basis of a contact assessment, an ablation progress assessment (e.g., a lesion formation/maturation analysis), and/or other assessment, as discussed below.

The tissue ablation electrode 24 may be any length and may have any number of the microelectrodes 26 positioned therein and spaced circumferentially and/or longitudinally thereabout. In some instances, the tissue ablation electrode 24 may have a length of between one (1) mm and twenty (20) mm, three (3) mm and seventeen (17) mm, six (6) mm and fourteen (14) mm, or other length. In one illustrative example, the tissue ablation electrode 24 may have an axial length of about eight (8) mm.

In some cases, the plurality of microelectrodes 26 may be spaced at any interval about the circumference of the tissue ablation electrode 24. In one example, the tissue ablation electrode 24 may include at least three microelectrodes 26 equally or otherwise spaced about the circumference of the tissue ablation electrode 24 and at the same or different longitudinal positions along the longitudinal axis of the tissue ablation electrode 24. In such instances, and/or other instances, the microelectrodes 26 may form at least the first bipolar microelectrode pair 28a, the second bipolar microelectrode pair 28b, and the third bipolar microelectrode pair 28c.

In some illustrative cases, the tissue ablation electrode 24 may have an exterior wall that at least partially defines an open interior region (not shown). The exterior wall may include one or more openings for accommodating one or more microelectrodes 26. Additionally, or alternatively, the tissue ablation electrode 24 may include one or more irrigation ports (not shown). Illustratively, the irrigation ports, when present, may be in fluid communication with an external irrigation fluid reservoir and pump (not shown) which may be used to supply fluid (e.g., irrigation fluid) to myocardial tissue to be or being mapped and/or ablated.

Illustrative catheters that may be used as the elongated member 12 may include, among other ablation and/or mapping catheters, those described in U.S. patent application Ser. No. 12/056,210 filed on Mar. 26, 2008, and entitled HIGH RESOLUTION ELECTROPHYSIOLOGY CATHETER, and U.S. Pat. No. 8,414,579 filed on Jun. 23, 2010, entitled MAP AND ABLATE OPEN IRRIGATED HYBRID CATHETER, which are both hereby incorporated by reference in their entireties for all purposes. Alternatively, or in addition, catheters that may be used as the elongated member 12 may include, among other ablation and/or mapping catheters, those described in U.S. Pat. No. 5,647,870 filed on Jan. 16, 1996, as a continuation of U.S. Ser. No. 206,414, filed Mar. 4, 1994 as a continuation-in-part of U.S. Ser. No. 33,640, filed Mar. 16, 1993, entitled MULTIPLE ELECTRODE SUPPORT STRUCTURES, U.S. Pat. No. 6,647,281 filed on Apr. 6, 2001, entitled EXPANDABLE DIAGNOSTIC OR THERAPEUTIC APPARATUS AND SYSTEM FOR INTRODUCING THE SAME INTO THE BODY, and U.S. Pat. No. 8,128,617 filed on May 27, 2008, entitled ELECTRICAL MAPPING AND CRYO ABLATING WITH A BALLOON CATHETER, where all of these references are hereby incorporated by reference in their entireties for all purposes.

In some illustrative instances, the processor 16 may be capable of detecting, processing, and/or recording or may be configured to detect, process, and/or record electrical signals, such as electrical signals within the heart (e.g., EGMs) or other electrical signals (e.g., within a patient or otherwise) via the elongated member 12. Based on the detected, processed, and/or recorded electrical signals, a physician may be able to assess contact at specific target area (e.g., target tissue) sites within the heart, and may be able to ensure any arrhythmia causing substrates have been electrically isolated by RF energy treatment from the RF ablation system 10.

The processor 16 may be capable of processing or may be configured to process the electrical signals of the output signals from the microelectrodes 26 and/or the ring electrodes 22. Based, at least in part, on the processed output signals from the microelectrodes 26 and/or the ring electrodes 22, the processor 16 may generate an output to a display (not shown) for use by a physician or other user.

In instances where an output is generated to a display and/or other instances, the processor 16 may be operatively coupled to or otherwise in communication with the display. Illustratively, the display may include various static and/or dynamic information related to the use of the RF ablation system 10. In one example, the display may include one or more of an image of the target area, an image of the elongated member 12, and information related to EGMs, which may be analyzed by the user and/or by a processor of the RF ablation system 10 to determine the existence and/or location of arrhythmia substrates within the heart, to determine the location of the elongated member 12 within the heart, and/or to make other determinations relating to use of the elongated member 12 and/or other elongated members.

As discussed, the RF ablation system 10 may include an indicator in communication with the processor 16. The indicator may be capable of providing an indication related to a feature of the output signals received from one or more of the electrodes of the elongated member 12.

The indicators may provide any type of indicating information to a user. For example, the indicators discussed herein may be pass or fail type indicators showing when a condition is present or is not present and/or may be progressive indicators showing the progression from a first level to a next level of a characteristic (e.g., contact assessment, lesion maturation, etc.).

In one example of an indicator, an indication to the clinician about a characteristic of the elongated member 12 and/or the myocardial tissue interacted with and/or being mapped may be provided on the display. In some cases, the indicator may provide a visual and/or audible indication to provide information concerning the characteristic of the elongated member 12 and/or the myocardial tissue interacted with and/or being mapped.

In an illustrative instance of providing visual indication of a characteristic of the elongated member 12 and/or the myocardial tissue interacted with or being mapped (e.g., indicating a level of a characteristic), the visual indication may take one or more visual colors or light forms. In some instances, a visual color or light indication on a display may be separate from or included on an imaged catheter on the display if there is an imaged catheter. Such a color or light indicator may include a progression of lights or colors that may be associated with various levels of each characteristic, where the levels may be proportional to the amplitude and/or frequency of an EGM. Alternatively, or in addition, an indicator indicating a level of the characteristics that may be proportional to an amplitude and/or frequency of an EGM may indicate in any other visual manner on a display and/or with any audible or other sensory indication, as desired.

In one example of an indicator, a color of at least a portion of an electrode of a catheter imaged on a screen of the display may change from a first color (e.g., red or any other color) when there is poor contact between the catheter and tissue to a second color (e.g., green or any other color different than the first color) when there is good contact between the catheter and the tissue and/or when RF energy application may be initiated after establishing good contact. Additionally or alternatively in another example of an indicator, when the amplitude and/or frequency spectra of the EGM stops changing and/or reaches a lesion maturation amplitude or frequency spectra threshold, a depicted color of an electrode on the imaged catheter may change colors to indicate a level of lesion maturation. In the examples above, the changing color/light or changing other indicator (e.g., a number, an image, a design, sound, etc.) may be located at or emanate from a position on the display other than on the imaged catheter, as desired.

The RF generator 14 may be capable of delivering and/or may be configured to deliver ablation energy to the elongated member 12 in a controlled manner in order to ablate target area sites identified by the processor 16. Ablation of tissue within the heart is well known in the art, and thus for purposes of brevity, the RF generator 14 will not be described in further detail. Further details regarding RF generators are provided in U.S. Pat. No. 5,383,874 filed Nov. 13, 1992, and entitled SYSTEMS FOR IDENTIFYING CATHETERS AND MONITORING THEIR USE, which is hereby incorporated by reference in its entirety for any purpose. Although the processor 16 and RF generator 14 may be shown as discrete components, these components or features of components may be incorporated into a single device.

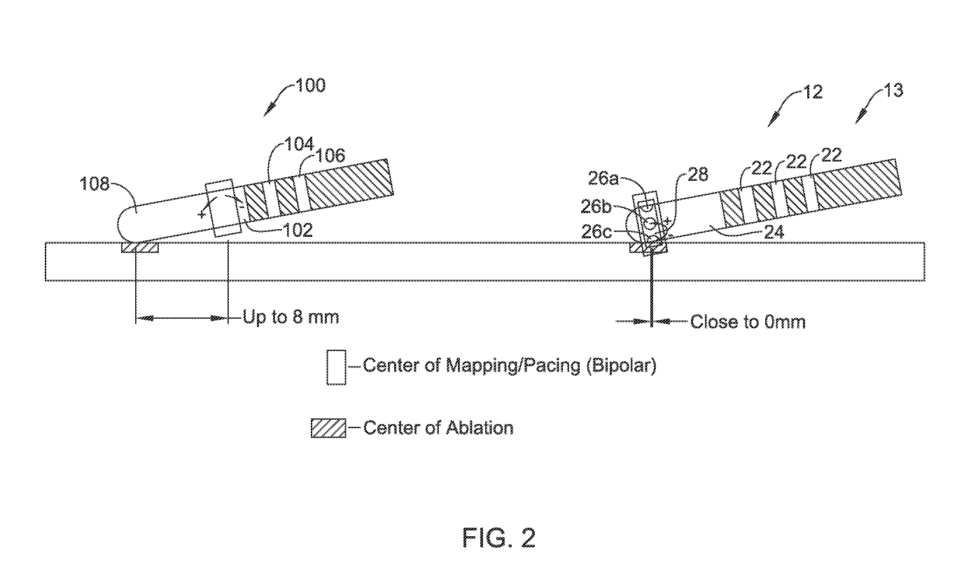

FIG. 2 is a schematic illustration showing a conventional ablation catheter 100 (e.g., an ablation catheter lacking any microelectrodes within the tissue ablation electrode) on the left and the elongated member 12 on the right. For cardiac mapping, the conventional ablation catheter 100 relies on conventional ring electrodes 102, 104, 106 disposed a distance from the ablation electrode 108. Such positioning of the ring electrodes 102, 104, 106 may result in a large distance between the center of the mapping and the center of the ablation on cardiac tissue. The elongated member 12, in contrast, may include the microelectrodes 26 in the tissue ablation electrode 24 to allow the center of mapping to be in substantially the same location as the center of ablation on the cardiac tissue.

### RF Ablation System Analyses

Based, at least in part, on its sensing capabilities, the elongated member 12 may be utilized to perform various diagnostic functions to assist the physician in ablation and/or mapping procedures, as referred to above and discussed further below. In one example, the elongated member 12 may be used to ablate cardiac arrhythmias, and at the same time provide real-time positioning information, real-time contact information, and real-time assessment of a lesion formed during ablation (e.g., during RF ablation). The presence of microelectrodes 26 at or about the tissue ablation electrode 24 and/or within the tip (e.g., at the distal tip) of the elongated member 12 may facilitate allowing a physician, in real time, to determine the position (e.g., orientation) and/or the contact force of the tissue ablation electrode relative to the tissue that is to be ablated or relative to any other feature. Additionally, the real-time assessment of the lesion may involve monitoring surfaces and/or tissue temperature at or around the lesion, monitoring changes in the electrogram signal amplitude and/or frequency, monitoring changes, if any, in impedance (e.g., an increase or decrease), monitoring direct and/or surface visualizations of the lesion site, and/or imaging a tissue site (e.g., using computed tomography, magnetic resonance imaging, ultrasound, etc.).

“Real-time”, as used herein and understood in the art, means during an action. For example, where one is monitoring frequency spectra in real time during an ablation at a target area, the frequency spectra is being monitored during the action of ablating at a target area.

In operation and when the elongated member 12 is within a patient and/or adjacent a target area, the elongated member 12 may sense electrical signals (e.g., EGM signals) from the patient or target area and relay those electrical signals to a physician (e.g., through the display of the RF ablation system 10 or other mechanism). Electrophysiologists and/or others may utilize EGM amplitude and/or EGM morphology to verify a position and/or orientation of the ablation catheter in a patient's anatomy, to determine a contact force level between the elongated member 12 and tissue adjacent the elongated member 12, to verify lesion formation in tissue adjacent the ablation catheter, and/or to verify or identify other characteristics related to the elongated member 12 and/or the target tissue or areas.

Determining EGM amplitude, which may be determined and/or measured in real-time by the processor 16, from peak-to-peak voltage of an EGM deflection, and/or from the ST segment of the P-QRS-T curve of an EGM reading or signal in a time domain, may quantify an intensity of an EGM signal and provide information relating to one or more target area characteristics. Illustratively, some target area characteristics include, but are not limited to, lesion maturation and contact force between the elongated member and tissue.

In some instances, empirical EGM data may be analyzed in a meaningful manner in real-time (e.g., during a typical electrophysiology procedure) by converting an EGM signal from the time domain to the frequency domain with the processor 16 and/or other processor. Such a conversion of an EGM signal from the time domain to the frequency domain may simplify EGM data interpretation and may allow for real-time insights related to contact assessments and/or lesion maturation assessments during an ablation procedure, among other real-time insights.

Illustratively, any technique, as desired, may be utilized to convert an EGM signal from the time domain, F(t), to a frequency domain, F(ω). In some examples, one or more of transforms (e.g., Fourier, Fast Fourier, Wavelet, Wigner-Ville), Welch's method, periodograms, etc. may be utilized to transform EGM signals from the time domain to the frequency domain. The signal processing techniques (e.g. mathematical operations, algorithms, etc.) utilized to transform signals from the time domain to the frequency domain are commonly known and understood by those skilled in the signal processing arts and, for brevity purposes, will not be reiterated here. In some instances, a Fast Fourier Transform (FFT) may be used, which may rapidly compute the transformation of an EGM signal from the time domain to the frequency domain.

In some instances, changes in a frequency spectrum may directly correspond to changes in one or more EGM characteristics (e.g., contact force, lesion maturation, etc.). Further, the changes in the frequency spectrum may be observed and utilized to adjust a particular therapy (e.g. ablation, etc.) used to treat the particular EGM characteristic (e.g., contact force, lesion maturation, etc.). However, the presence of noise in a frequency spectrum may prevent and/or limit accurate observation of relevant changes in the frequency spectrum. Therefore, in some instances, “windowing” may be utilized to reduce noise in a frequency spectrum of an EGM signal.

For example, in the time domain, EGM signals may include a P-QRS-T wave and a relatively “flat line” connecting sequential P-QRS-T waves. When such EGM signals are transformed from the time domain to the frequency domain, the flat line connecting sequential P-QRS-T waves may introduce a confounding artifact into the frequency spectrum. As a result, windowing may be utilized to convert only a portion of the EGM signal to the frequency domain, such that some of the artifacts present in a typical EGM signal in the frequency domain may be eliminated from the analyzed EGM signal. In one illustrative example, a window may, among other windowing periods, include any period less than a continuous EGM signal that includes a single activation of an EGM signal, a period that includes a predetermined time period before (e.g,., 1 millisecond (ms), 5 ms, 10, ms, 20, ms, 30 ms, 40, ms, 50 ms, etc.) and/or after (e.g., 10 ms, 50 ms, 100 ms, 150, ms, 200 ms, 250 ms, 500 ms, etc.) an activation of an EGM signal, a period that is configured to includes the QRS segment of the P-QRS-T wave, and/or any other window configured to eliminate some noise of an EGM signal.

In some instances, a curved, non-rectangular windowing function may be used to mitigate artifacts introduced into the frequency domain representation of the signal as a result of windowing. These artifacts are well understood and documented by those familiar with signal processing and frequency analysis techniques. Examples of such curved, non-rectangular windowing functions include, but are not limited to, Hamming windows, Hann windows, and raised cosine windows.

As referred to, real-time data in the time domain and/or frequency domain for EGM signals may facilitate assessments of contact between the tissue ablation electrode 24 and the target tissue based on EGM related characteristics. Illustratively, as contact pressure between the elongated member 12 and the cardiac tissue increases, an “injury current” may be visible on the EGM from changes to an ST segment of a P-QRS-T curve of the EGM and/or from changes in the frequency of the EGM over time.

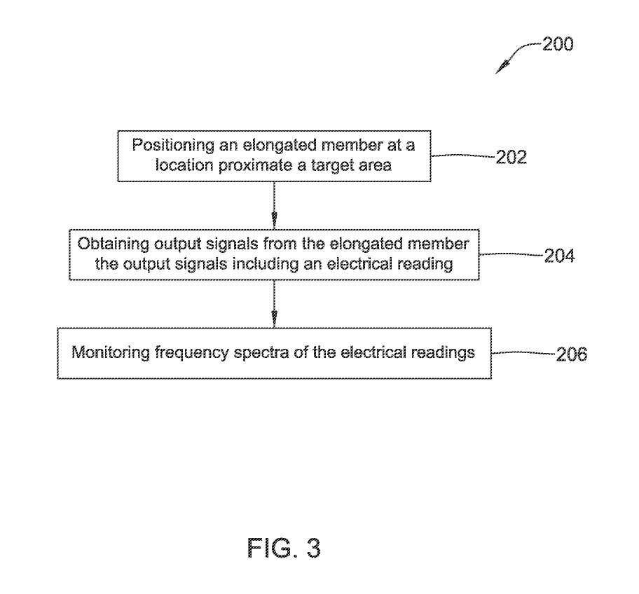

Illustratively, real time data in the time domain and/or frequency domain may be used in a method 200 depicted in FIG. 3. In the method 200, a distal portion of the elongated member 12 may be positioned 202 at a location proximate a target area or target tissue. The processor 16 of or in communication with the RF ablation system 10 may obtain 204 output signals from the electrodes (e.g., the ablation electrode 24, microelectrodes 26, and/or other electrodes) of the elongated member 12 adjacent a target area or tissue (e.g., output signals from each bipolar microelectrode pair 28 of the elongated member 12 or other electrode of elongated member 12).

The output signals obtained from the electrodes of the elongated member 12 may comprise an electrical signal or reading from the heart (e.g., an EGM signal or reading, or other electrical signal). The electrical signals of electrodes may be obtained, read, and/or analyzed in the time domain and/or the frequency domain. In instances when the electrical signals of the obtained output signals are received in the time domain, but are to be analyzed in the frequency domain, the processor 16 may be utilized to convert the electrical signals from the time domain to the frequency domain. Once the electrical signals of the obtained output signals are in the frequency domain, the frequency spectra of the electrical signal (e.g., the EGM) may be monitored 206.

In some instances, an amplitude of the electrical signal (e.g., an amplitude of the EGM signal) of the obtained output signals may be identified with the processor 16 and/or other processor of, or in communication with, the RF ablation system 10. In one illustrative example and as discussed above, the amplitude of an electrical signal of the obtained output signals may be the amplitude of the ST-segment of a P-QRS-T wave of an EGM in the time domain. Additionally or alternatively, other amplitudes of an electrical signal may be identified and/or utilized, as desired.

Once an amplitude and frequency spectra of the electrical signal from electrodes of the elongated member 12 are identified or determined, levels of one or more characteristics that are proportional to one or more of the identified amplitudes and frequency spectra may be determined and/or monitored. In one illustrative example and as discussed above, the one or more characteristics may include, but are not limited to, contact force between the elongated member 12 and a target area (e.g., a target tissue or other target area) and ablation progress (e.g., lesion maturation or other metric of ablation progress).

As discussed, a level of contact force between the elongated member (e.g., the elongated member 12) and the target area (e.g., target tissue or other target area) or other area may be determined from one or more of the identified amplitude and frequency spectra of the electrical signal (e.g., EGM) of the obtained output signals. In some instances of determining a level of contact force between the elongated member 12 and the target area, the level of contact force may be determined in real time, for example, while positioning the distal portion of the elongated member proximate the target area, while mapping a target area or other object, while applying ablation energy to a target area, and/or while performing any other action with the elongated member.

When determining the level of contact force between the elongated member (e.g., elongated member 12) and the target area, the determination may include one or more of comparing the amplitude of the electrical signal to one or more amplitude thresholds with the processor 16 and comparing the monitored frequency spectra to one or more frequency thresholds with the processor 16. The amplitude threshold and/or the frequency threshold may be predetermined thresholds or thresholds determined from baseline amplitude and/or frequency readings at the beginning of or during an ablating or mapping process or procedure.

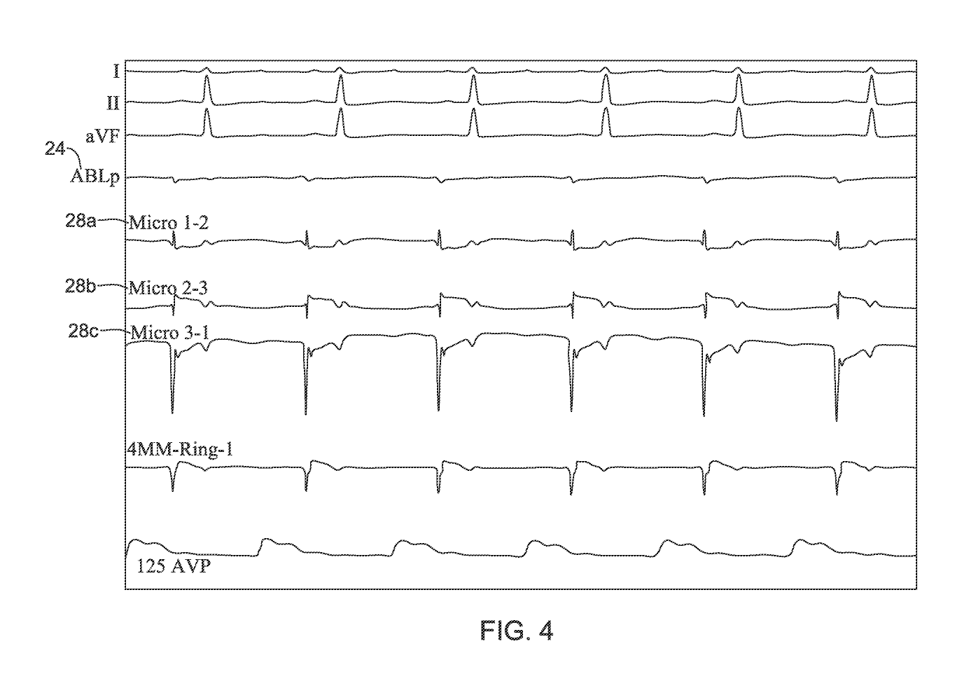

In one illustrative example of determining the level of contact force between the elongated member (e.g., the elongated member 12) and the target area, when an amplitude of the electrical signal is above an amplitude threshold and the frequency spectra of the electrical signal is below a frequency spectra threshold, the determined level of contact force may be a first level of contact force. In the illustrative example, when the amplitude of the electrical signal is above the amplitude threshold and the frequency spectra of the electrical signal is above the frequency threshold, the determined level of contact force may be a second level of contact force that is less than the first level of contact force. Further in the illustrative example, when the amplitude of the electrical signal is below the amplitude threshold and the frequency spectra of the electrical signal is below the frequency threshold, the determined level of contact force may be a third level of contact force that is less than the second level of contact force. Alternatively or in addition, the level of contact force may be determined in any other manner using the amplitude of the electrical signal, the frequency of the electrical signal, or both the amplitude and the frequency of the electrical signal. See discussion of FIGS. 4-6 below for a discussion of interpreting electrical signal amplitudes and frequencies.

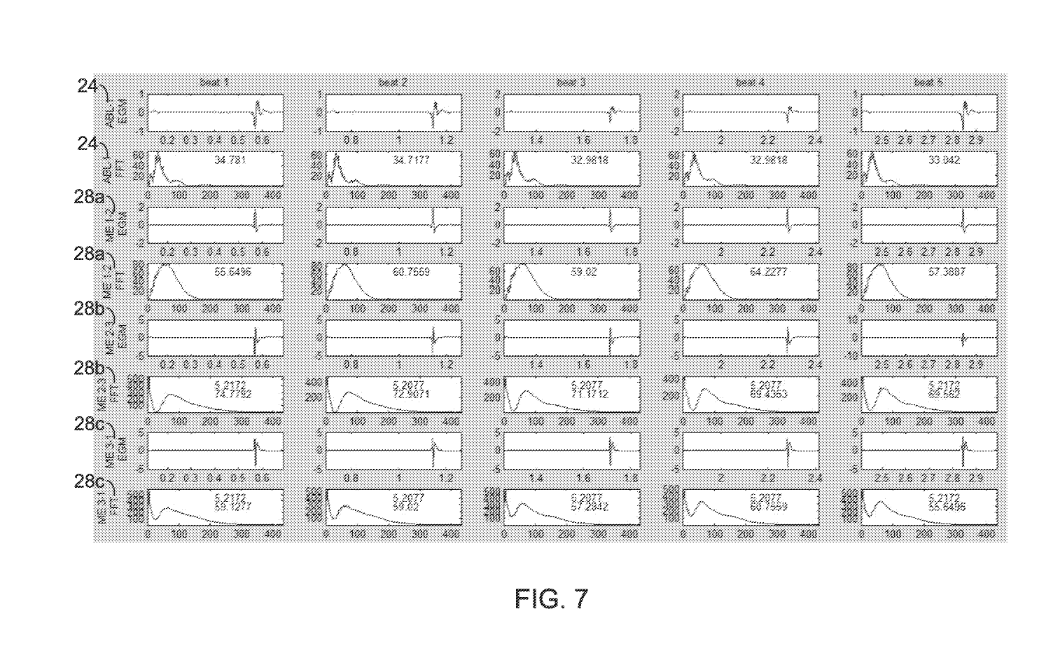

Once it has been determined the distal end of the elongated member 12 is in a desired contact with a target area (e.g., a target tissue or other target area), or at any other time, ablation energy may be applied to the target area with an ablation electrode 24. In some instances, during the application of ablation energy to the target area, the processor 16 may determine the progress of the ablation (e.g., a level of lesion maturation or other metric of the progress of the ablation) in real time based on at least one or more of the identified amplitudes and the frequency spectra of the electrical signal of the obtained output signal from the electrodes (e.g., amplitudes and frequency spectra of the EGM signal from the bipolar microelectrode pairs). See discussion of FIGS. 7-9 for further discussion of ablation progress assessment (e.g., lesion maturation assessment)

During the method 200 of using the RF ablation system 10 or other method, a level of one or more of the characteristics that are proportional to one or more of the amplitude and frequency spectra of the electrical signals (e.g., EGMs) of the obtained output signals from the electrodes of the elongated member may be indicated. For example, as discussed above, the level of one or more of the characteristics may be displayed visually on a display, may be indicated by an audible indicator, and/or may be indicated in any other manner.

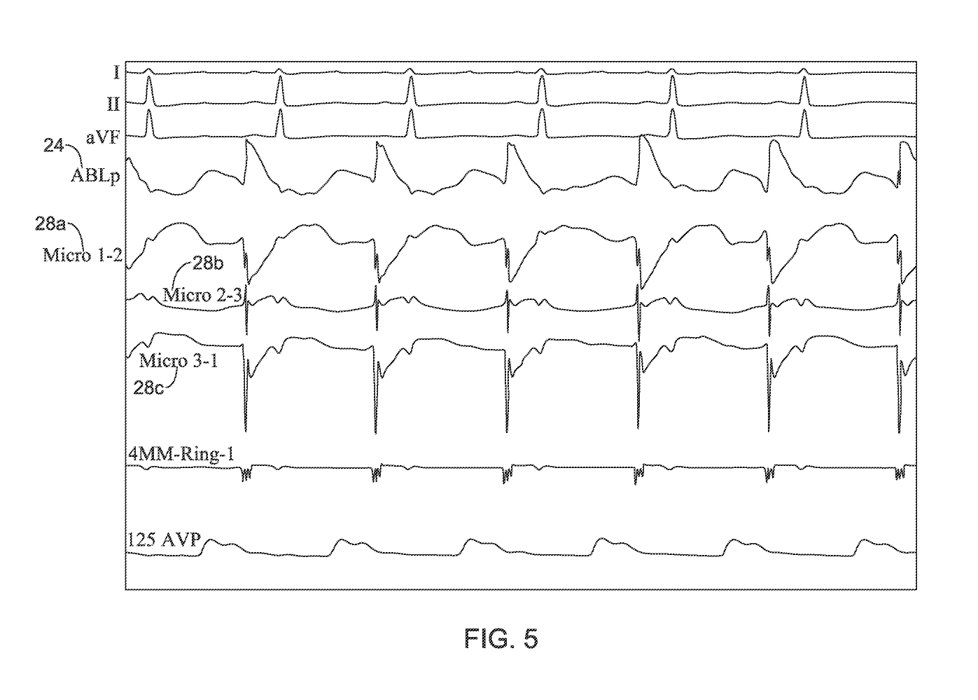

FIGS. 4 and 5 illustrate EGMs in the time domain depicting levels of contact force at or adjacent particular bipolar microelectrode pairs. These figures show several EGMs, for example, one EGM is depicted for the ablation electrode 24 and one EGM is depicted for each bipolar microelectrode pair 28, among others.

FIG. 4 depicts EGMs in the time domain that illustrate light contact between the distal end 13 of the elongated member 12 and cardiac tissue. Light contact between the third bipolar microelectrode pair 28c (e.g., the bipolar microelectrode pair 28 between the third microelectrode 26c and the first microelectrode 26a) and the cardiac tissue may be observed from the normal to slightly depressed ST-segment of the depicted P-QRS-T curve of the EGM of the third bipolar microelectrode pair 28c, relative to the ST-segments of the EGMs of the other bipolar microelectrode pairs 28a, 28b.

FIG. 5 is an EGM in the time domain that illustrates strong contact between the distal end 13 of the elongated member 12 and cardiac tissue. Strong contact between the third bipolar microelectrode pair 28c and the cardiac tissue may be observed from significant alteration of the ST-segment of the P-QRS-T curve of the EGM of the third bipolar microelectrode pair 28c due to one or more physical changes in the cardiac tissue from the force of contact by the elongated member 12, relative to the ST-segments of the EGMs of the other bipolar microelectrode pairs 28a, 28b.

As referred to above, received EGM signals may be analyzed in the frequency domain in addition to or as an alternative to analyses of EGMs in the time domain. By utilizing FFT or other transformation techniques to transform the EGM signal from the time domain to the frequency domain, it may be possible to quantify the injury current present (e.g., which may be proportional to a level of an applied force) when applying a force to the target tissue via the elongated member 12 through observation of a frequency spectra or a characteristic of a frequency spectra (e.g., a dominant or maximum frequency, a power weighted mean, a range of frequency, etc.) of the EGM signal.

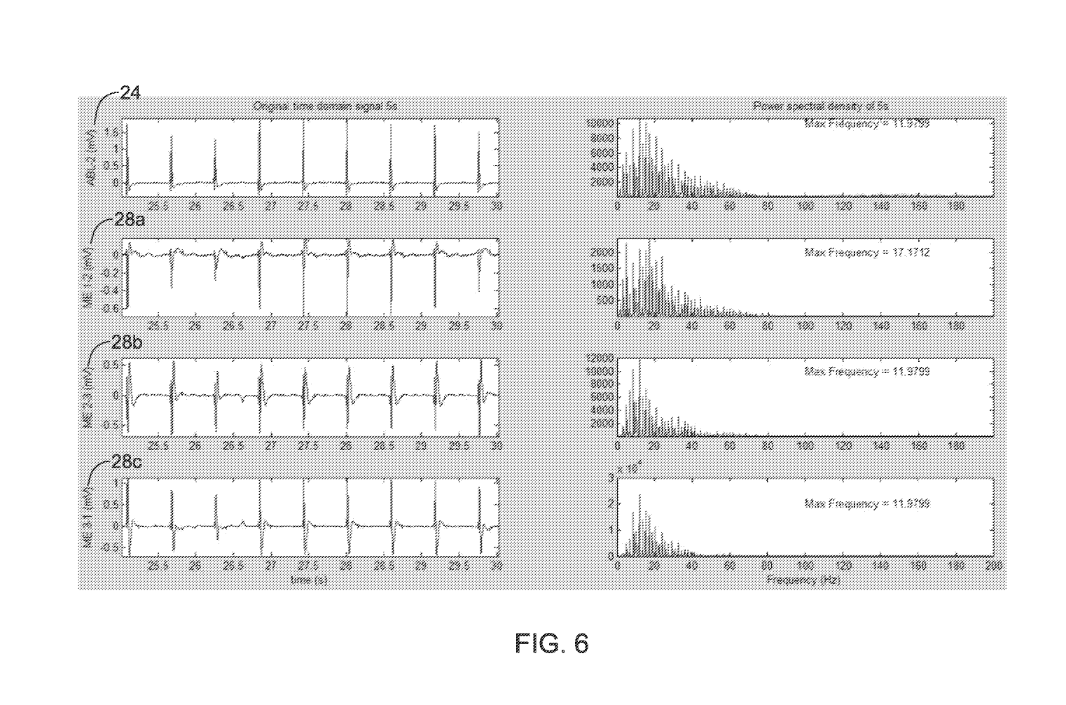

FIG. 6 depicts an illustrative example of EGM signals in the time domain for the tissue ablation electrode 24 and the three bipolar microelectrode pairs 28 (e.g., the first microelectrode pair 28a, the second microelectrode pair 28b, and the third microelectrode pair 28c), along with the corresponding EGM signals in the frequency domain. The plots of the EGM signals in the time domain are five (5) seconds of the respective EGM signal. The plots of the EGM signals in the frequency domain cover frequencies from zero (0) to two-hundred (200) hertz (Hz). The dominant or maximum frequency for each bipolar microelectrode pair 28 is included in each plot of the EGM signal in the frequency domain. Illustratively, a dominant or maximum frequency is the frequency of the frequency spectra with a greatest amplitude or power.

As may be seen from the left side of the example depicted in FIG. 6, the bipolar microelectrode pair 28 with the highest EGM amplitude relative to the other depicted EGM amplitudes, as determined from the elevation of the ST-segment of the P-QRS-T curve and/or from the greatest peak-to-peak distance of the curve, is the third bipolar microelectrode pair 28c. From this amplitude information, it may be determined the third bipolar microelectrode pair 28c is in the best contact with the cardiac tissue.

As may be seen from the right side of the example depicted in FIG. 6, the dominant frequency for the third bipolar microelectrode pair 28c is about 11.98 Hz, which corresponds to a 0.083 second period of the EGM signal. The 0.083 second period approximately corresponds to the period of the EGM signal in the time domain name from a peak of the R-wave to a peak of the T wave.

As referred to above, determining a level of contact force or pressure an elongated member 12 is applying to the tissue may be determined from analyzing an EGM plot in the time domain and an EGM plot in the frequency domain, such as those in FIG. 6. For example, in addition to, or as an alternative to, comparing EGM amplitude and frequency to thresholds, an observed relatively large amplitude of an EGM in the time domain and an observed relatively low range of frequency spectra, relatively low power weighted mean frequency, or relatively low maximum frequency may indicate there is a high contact force between the elongated member 12 and the tissue; an observed relatively large amplitude of an EGM in the time domain and an observed relatively high range of frequency spectra, relatively high power weighted mean frequency, or relatively high maximum frequency may indicate there is a stable contact force between the elongated member 12 and the tissue; an observed relatively low amplitude of an EGM in the time domain and an observed relatively low range of frequency spectra, relatively low power weighted mean frequency, or relatively low maximum frequency may indicate there is poor contact force between the elongated member 12 and the tissue. The relative high and low amplitudes, ranges of frequency spectra, power weighted mean frequencies, and/or maximum or dominant frequencies may be relative to the EGMs of the other bipolar microelectrode pairs, relative to sequential EGM signals, relative to thresholds determined before, at, or after initialization of an ablation or mapping process, and/or relative to any other metric depending on the context of the EGM signals and the characteristic being analyzed.

FIGS. 7-9 depict time domain and frequency domain images of EGM signals over time from an elongated member 12 having a tissue ablation electrode 24, a first microelectrode 26a, a second microelectrode 26b, and a third microelectrode 26c. As referred to above, lesion maturation during ablation may be assessed with real-time frequency spectral analysis. For example, from monitoring the frequency of an EGM signal received from an elongated member 12 over time, as shown in FIGS. 7-9, it may be possible to determine lesion development or maturation.

In each figure of FIGS. 7-9, the first row depicts plots of windows including an individual beat in the time domain of the EGM signal from a conventional bipole of the tissue ablation electrode 24, the second row depicts plots of the frequency domain for the conventional bipole of the tissue ablation electrode, the third row depicts plots of windows including individual beats in the time domain of the EGM signal from a first bipolar microelectrode pair 28a between the first microelectrode 26a and the second microelectrode 26b, the fourth row depicts plots of the frequency domain of the EGM signal from the first bipolar microelectrode pair 28a, the fifth row depicts plots of windows including individual beats in the time domain of the EGM signal from a second bipolar microelectrode pair 28b between the second microelectrode 26b and the third microelectrode 26c, the sixth row depicts plots of the frequency domain of the EGM signal from the second bipolar microelectrode pair 28b, the seventh row depicts plots of windows including individual beats in the time domain of the EGM signal from a third bipolar microelectrode pair 28c between the third microelectrode 26c and the first microelectrode 26a, and the eighth row depicts plots of the frequency domain of the EGM signal from the third bipolar microelectrode pair 28c.

FIG. 7 depicts time domain and frequency domain plots, as discussed above, of an EGM signal for the four bipolar pairs for three (3) seconds before an ablation of a target cardiac tissue occurs. The plots in rows 1, 3, 5, and 7 show the EGM signal in the time domain for zero (0) seconds to three (3) seconds. The plots in rows 2, 4, 6, and 8 show the EGM signal in the frequency domain between zero (0) and four hundred (400) hertz (Hz) during the same time period.

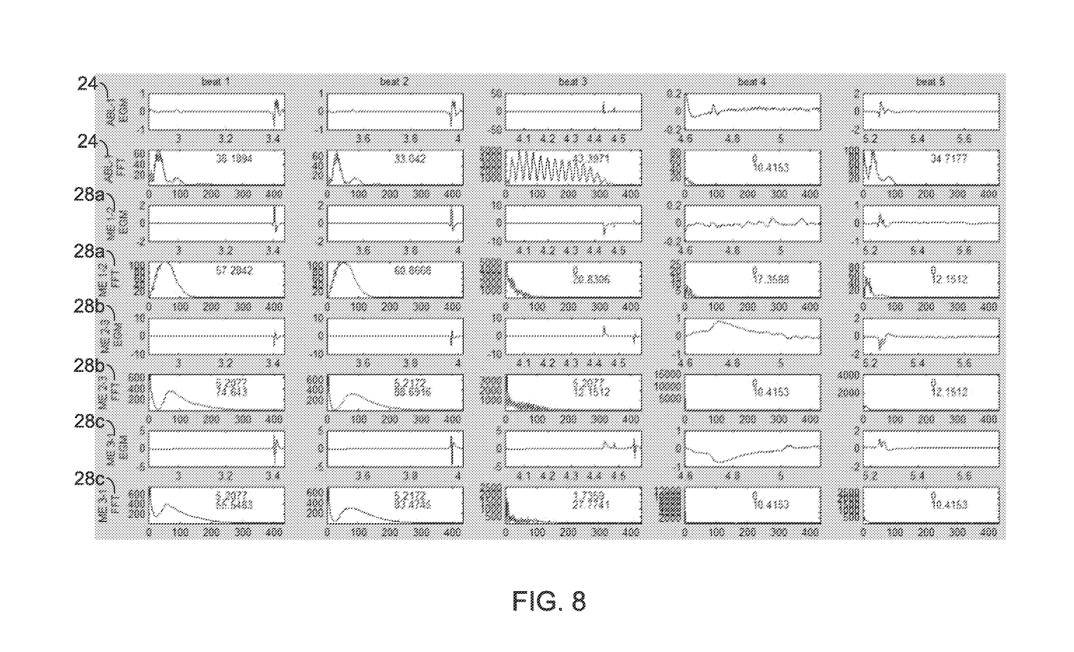

FIG. 8 depicts time domain and frequency domain plots, as discussed above, of an EGM signal for the four bipolar pairs for the time in which radio frequency energy of the tissue ablation electrode 24 has been turned on. The plots in rows 1, 3, 5, and 7 show the EGM signal in the time domain for seconds three (3) to six (6) of the process. The plots in rows 2, 4, 6, and 8 show the EGM signal in the frequency domain between zero (0) and four hundred (400) Hz during the same time period.

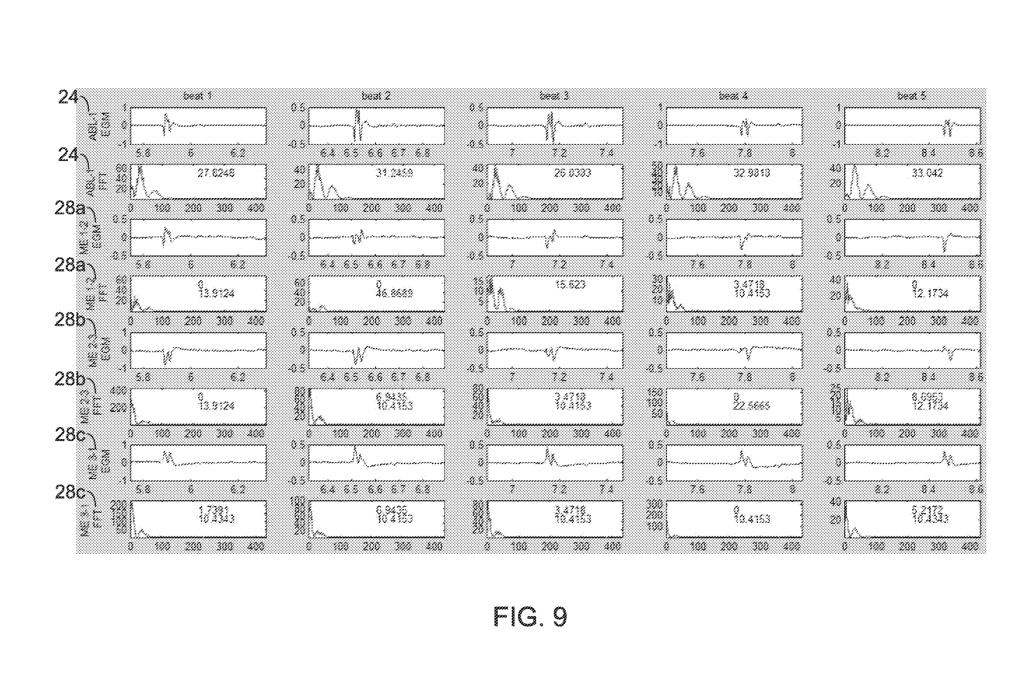

FIG. 9 depicts time domain and frequency domain plots, as discussed above, of an EGM signal for the four bipolar pairs for three (3) seconds of initial ablation. The plots in rows 1, 3, 5, and 7 show the EGM signal in the time domain for seconds 5.8 to 8.8 of the process. The plots in rows 2, 4, 6, and 8 show the EGM signal in the frequency domain between zero (0) and four hundred (400) Hz during the same time period.

In addition to, or as an alternative to, analyzing frequency spectra of EGM signals in view of frequency spectra ranges or dominant (or maximum) frequency as depicted in FIGS. 7-9, analyses of the frequency spectra of EGMs may be analyzed in view of characteristics other than frequency spectra ranges and dominant (or maximum) frequency. For example, analyses of the frequency plots of EGMs may be analyzed in view of one or more other filtering techniques including, but not limited to, frequency envelopes of the EGM signals, combinations of frequency envelopes of the EGM signals, sampling windows (as discussed above), power weighted mean frequency, and/or other or further filtering of the EGM signals, and/or any other analyses to determine one or more characteristics of an EGM signal.

Illustratively, changes in the power weighted mean (PWM) of an EGM signal's frequency spectra may be analyzed as it relates to an EGM characteristic. A PWM for each EGM signal or windowed EGM signal may be calculated with the following equation:

PWM=Σ(Frequencyᵢ*Powerᵢ)/ΣPowerᵢ (1)

where ““Powerᵢ” represents the power at each frequency bin, “Frequencyᵢ”, included in the EGM's frequency spectrum.

The PWM may be presented to physicians in one or more manners. In one example, a PWM may be presented to a physician as a line on a depiction of a frequency spectrum, where the line is configured to stick out from the frequency spectrum (e.g., through being a dotted line, a color that is easily distinguishable from a color of the frequency spectrum, etc.) Alternatively, or in addition, the PWM may be presented to a physician as a number on a display or audibly recited, on a display as a line graph showing the PWM over time, an audible or displayed indication of lesion maturation over time based on a PWM being calculated in the background, and/or through one or more other indications related to a PWM of the frequency spectra of an EGM signal.

By monitoring the power weighted mean of the frequency spectra of an EGM signal, the effects of signal noise and outlier frequencies on the EGM signal may be mitigated, as compared to analyses of frequency spectra ranges and/or dominant frequency, only. Such mitigation of artifacts may result in a more meaningful or distinct EGM signal in the frequency domain over time than if only a maximum or dominant frequency and/or frequency spectra range of the EGM signal were monitored. As can be understood from the above discussion, lesion maturation may be related to the amount of ablation energy applied to a lesion over time. Further, the degree of lesion maturation may be observed via changes in the frequency spectrum of an EGM signal. Further, a physician and/or processing system may be able to measure how effective their application of RF energy has been from observed changes in the frequency spectrum of an EGM signal and thus, may be able to tailor (e.g. reduce) the applied energy. Reducing the amount of energy applied during ablation therapy may lessen the chance of over-applying ablation energy to an already-mature lesion, which may improve safety during an ablation procedure.

In some instances the frequency spectrum of an EGM signal may begin to be dominated by a baseline signal with low frequency noise as ablation energy is applied. This low frequency noise may interfere with a physician and/or processing system's ability to accurately monitor lesion maturation. As a result, the physician and/or processing system may over-apply ablation energy thereby resulting in an over-developed lesion. However, utilizing the PWM of the EGM signal (via weighting the frequencies by their respective power, as discussed above) in the frequency domain (instead of or in addition to monitoring frequency spectra ranges or dominant frequency) may allow for monitoring EGM signals in the frequency domain while reducing the effect of baseline signal noise and/or other noise. Therefore, as compared to monitoring frequency spectra ranges or dominant frequencies, a physician or processor utilizing the PWM may realize an increase in the efficacy of ablation (e.g., application of RF energy). In one example, such an increase in the efficacy of ablation may allow a physician or processor to determine earlier in the ablation process when an appropriate amount of RF energy has been applied and the resulting lesion has been fully formed. The increase in the efficacy of ablation may be made possible because the analysis of PWM is more sensitive to changes in the EGM signal than the analyses of frequency spectra ranges and/or dominant frequencies. This is due, at least in part, to a reduction of the effect of outlier frequencies on the frequency spectra.

In an example similar to the example depicted in FIGS. 7-9, a window including a single and/or multiple beats of an EGM signal may be monitored in the time domain and/or frequency domain. These time sequential windows may be monitored for changes in the EGM signal over time. Illustratively, instead of monitoring a frequency spectra range or dominant frequency (as shown in the example of FIGS. 7-9), a PWM of the frequency spectra of a chosen window (e.g., a beat of the EGM signal or a portion of a beat of an EGM signal) may be monitored over time sequential windows. Such monitoring of the PWM of the EGM signal may further reduce the effect of outlier frequencies on a frequency spectra analysis, and therefore, may allow a processor and/or a physician to more precisely determine when an adequate lesion has been formed.

Illustratively (and as may be inferred from FIGS. 7-9) the maturation of a lesion may directly correspond and be observed from reduced EGM amplitude and frequency spectra (e.g., frequency spectra ranges, PWM frequencies, and/or dominant frequency) over time. Further, analyzing PWMs of EGM signals in the frequency domain, real-time tracking of EGM morphology, and EGM amplitude in the time domain may facilitate and assist in the monitoring of lesion creation and/or may allow for an ablation treatment to be altered in real-time in response to a rate at which a lesion is maturing.

For example, it has been determined that in thin target tissues a transmural lesion may be formed in less than ten (10) seconds. The real time frequency analysis discussed herein may shorten ablation times while mitigating the potential for complications caused by stream pops, thrombus formations, and/or other thermal or other injuries adjacent to a target cardiac tissue, all of which may occur from a less precise analysis of lesion maturation.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

### Additional Examples

In a first example, a system may include: an elongated member having a distal portion, the distal portion of the elongated member comprising one or more electrodes; a radio frequency generator operatively coupled to one or more of the one or more electrodes for generating energy to be conveyed to one or more of the one or more electrodes, the processor is capable of: obtaining output signals from one or more of the electrodes, one or more of the output signals comprises an electrogram (EGM) reading; and monitoring changes in frequency spectra of the EGM in the frequency domain.

In a second example, the system of example one, wherein the processor is capable of converting the EGM from a time domain to a frequency domain.

In a third example, the system of either one of example one or example two, wherein monitoring changes in frequency spectra of the EGM in a frequency domain includes monitoring time sequential windows for changes in the EGM over time.

In a fourth example, the system of any one of examples one through three, further including: an indicator in communication with the processor, the indicator is capable of providing an indication related to the monitored frequency spectra.

In a fifth example, the system of any of examples one through four, wherein the processor is capable of: identifying an amplitude of one or more of the EGMs; and determining a level of one or more characteristics that are proportional to one or more of the identified amplitudes and one or more of the frequency spectra.

In a sixth example, the system of example five wherein the one or more characteristics comprise one or more of contact force between the elongated member and a target tissue and lesion maturation.

In a seventh example, the system of either one of example five or example six, wherein an indicator indicates the level of the one or more characteristics that are proportional to one or more of the identified amplitudes and the frequency spectra.

In an eighth example, the system of any one of examples one through seven, where the distal portion of the elongated member comprises: a tissue ablation electrode configured to apply ablation energy to a target tissue; and a plurality of microelectrodes distributed about the tissue ablation electrode and electrically isolated therefrom, the plurality of microelectrodes defining a plurality of bipolar microelectrode pairs, each bipolar microelectrode pair is capable of generating an output signal.

In a ninth example, the system of any one of examples one through eight, wherein monitoring changes in frequency spectra of the EGM in a frequency domain includes monitoring a power weighted mean of the frequency spectra of the EGM.

In a tenth example, a method comprises: positioning a distal portion of an elongated member at a location proximate a target tissue, the distal portion of the elongated member comprising: a tissue ablation electrode configured to apply ablation energy to the target tissue; and a plurality of microelectrodes distributed about the tissue ablation electrode and electrically isolated therefrom, the plurality of microelectrodes defining a plurality of bipolar microelectrode pairs, each bipolar microelectrode pair capable of generating an output signal; obtaining output signals from one or more of the bipolar microelectrode pairs, one or more of the output signals comprises an electrogram (EGM) reading; and monitoring frequency spectra of the EGM in a frequency domain.

In an eleventh example, the method of example ten, wherein the EGM readings of the output signals are in a time domain, the method further comprises: converting the EGM readings from the time domain to the frequency domain.

In a twelfth example, the method of either one of example ten or example eleven, further including: identifying an amplitude of one or more of the EGMs in a time domain; determining a level of one or more characteristics that are proportional to one or more of the identified amplitudes and the frequency spectra, the one or more characteristics comprise one or more of contact force between the elongated member and the target tissue and lesion maturation.

In a thirteenth example, the method of any one of examples ten through twelve, further including: applying ablation energy to the target tissue with the tissue ablation electrode; and determining lesion maturation on the target tissue during the application of ablation energy to the target tissue with the tissue ablation electrode, wherein lesion maturation is proportional to one or more identified amplitudes of the EGMs and the monitored frequency spectra of the EGMs.

In a fourteenth example, the method of any one of examples ten through thirteen, further including: determining a level of contact force between the elongated member and the target tissue, wherein determining a level of contact force comprises one or more of: comparing the identified amplitudes to one or more amplitude thresholds; and comparing the monitored frequency spectra to one or more frequency thresholds.

In a fifteenth example, the method of any of examples ten through fourteen, further including: determining a level of contact force between the elongated member and the target tissue; wherein determining a level of contact force between the elongated member and the target tissue occurs while positioning the distal portion of the elongated member proximate the target tissue.

In a sixteenth example, the method of any one of examples ten through fifteen, further including: indicating on a display a level of one or more characteristics that are proportional to one or more of an identified amplitude of the EGMs and the frequency spectra of the EGMs, the one or more characteristics comprise one or more of a contact force between the elongated member and the target tissue and lesion maturation.

In an seventeenth example, a system including: an elongated member having a distal portion, the distal portion of the elongated member comprising: a tissue ablation electrode configured to apply ablation energy to a target tissue; and a plurality of microelectrodes distributed about the tissue ablation electrode and electrically isolated therefrom, the plurality of microelectrodes defining a plurality of bipolar microelectrode pairs, each bipolar microelectrode pair capable of generating an output signal; a radio frequency generator operatively coupled to the tissue ablation electrode for generating the energy to be conveyed to the tissue ablation electrode; a processor operatively coupled to one or more of the tissue ablation electrode and the microelectrodes, and while one or more of positioning the distal portion of the elongated member adjacent the target tissue and applying ablation energy to the target tissue, the processor is capable of: obtaining output signals from each of the bipolar microelectrode pairs, one or more of the output signals comprises an electrogram (EGM) reading; and monitoring changes in frequency spectra of the EGMs in a frequency domain; and an indicator indicating a level of lesion maturation that is proportional to changes in the frequency spectra of the EGM.

In a eighteenth example, the system of example seventeen, wherein the indicator is capable of indicating the level of lesion maturation during application of ablation energy to the target tissue.

In a nineteenth example, the system of either one of example seventeen or example eighteen, wherein the processor is capable of determining a level of one or more characteristics that are proportional to the frequency spectra, the one or more characteristics comprise contact force between the elongated member and the target tissue.

In a twentieth example, the system of any one of examples seventeen through nineteen, wherein the processor is capable of converting the EGM readings from a time domain to the frequency domain.

What is claimed is:

1. A system comprising:
an elongated member having a distal portion, the distal portion of the elongated member comprising one or more electrodes;
a radio frequency generator operatively coupled to one or more of the one or more electrodes for generating energy to be conveyed to one or more of the one or more electrodes;
a processor operatively coupled to one or more of the one or more electrodes, the processor is capable of:
obtaining output signals from one or more of the electrodes, one or more of the output signals comprises an electrogram (EGM) reading; and
monitoring changes in frequency spectra of the EGM in a frequency domain.

2. The system of claim 1, wherein the processor is capable of converting the EGM readings from a time domain to the frequency domain.

3. The system of claim 1, wherein monitoring changes in frequency spectra of the EGM in a frequency domain includes monitoring time sequential windows for changes in the EGM over time.

4. The system of claim 1, further comprising:
an indicator in communication with the processor, the indicator is capable of providing an indication related to the monitored frequency spectra.

5. The system of claim 1, wherein the processor is capable of:
identifying an amplitude of one or more of the EGMs; and
determining a level of one or more characteristics that are proportional to one or more of the identified amplitudes and one or more of the frequency spectra.

6. The system of claim 5, wherein the one or more characteristics comprise one or more of contact force between the elongated member and a target tissue and lesion maturation.

7. The system of claim 5, wherein an indicator indicates the level of the one or more characteristics that are proportional to one or more of the identified amplitudes and the frequency spectra.

8. The system of claim 1, where the distal portion of the elongated member comprises:
a tissue electrode configured to apply RF energy to a target tissue; and
a plurality of microelectrodes distributed about the tissue electrode and electrically isolated therefrom, the plurality of microelectrodes defining a plurality of bipolar microelectrode pairs, each bipolar microelectrode pair is capable of generating an output signal.

9. The system of claim 1, wherein monitoring changes in frequency spectra of the EGM in a frequency domain includes monitoring a power weighted mean of the frequency spectra of the EGM.

10. A method, the method comprising:
positioning a distal portion of an elongated member at a location proximate a target tissue, the distal portion of the elongated member comprising:
a tissue electrode configured to apply RF energy to the target tissue; and
a plurality of microelectrodes distributed about the tissue electrode and electrically isolated therefrom, the plurality of microelectrodes defining a plurality of bipolar microelectrode pairs, each bipolar microelectrode pair capable of generating an output signal;
obtaining output signals from one or more of the bipolar microelectrode pairs, one or more of the output signals comprises an electrogram (EGM) reading; and
monitoring frequency spectra of the EGM in a frequency domain.

11. The method of claim 10, wherein the EGM readings of the output signals are in a time domain, the method further comprises:
converting the EGM readings from the time domain to the frequency domain.

12. The method of claim 10, further comprising:
identifying an amplitude of one or more of the EGMs in a time domain;
determining a level of one or more characteristics that are proportional to one or more of the identified amplitudes and the frequency spectra, the one or more characteristics comprise one or more of contact force between the elongated member and the target tissue and lesion maturation.

13. The method of claim 10, further comprising:
applying RF energy to the target tissue with the tissue electrode; and
determining lesion maturation on the target tissue during the application of RF energy to the target tissue with the tissue electrode, wherein lesion maturation is proportional to one or more identified amplitudes of the EGMs and the monitored frequency spectra of the EGMs.

14. The method of claim 10, further comprises:
determining a level of contact force between the elongated member and the target tissue, wherein determining a level of contact force comprises one or more of:
comparing the identified amplitudes to one or more amplitude thresholds; and
comparing the monitored frequency spectra to one or more frequency thresholds.

15. The method of claim 10, further comprising:
determining a level of contact force between the elongated member and the target tissue;
wherein determining a level of contact force between the elongated member and the target tissue occurs while positioning the distal portion of the elongated member proximate the target tissue.

16. The method of claim 10, further comprising:
indicating on a display a level of one or more characteristics that are proportional to one or more of an identified amplitude of the EGMs and the frequency spectra of the EGMs, the one or more characteristics comprise one or more of a contact force between the elongated member and the target tissue and lesion maturation.

17. A system comprising:
an elongated member having a distal portion, the distal portion of the elongated member comprising:
an electrode configured to apply RF energy to a target tissue; and
a plurality of microelectrodes distributed about the electrode and electrically isolated therefrom, the plurality of microelectrodes defining a plurality of bipolar microelectrode pairs, each bipolar microelectrode pair capable of generating an output signal;
a radio frequency generator operatively coupled to the electrode for generating the energy to be conveyed to the electrode;
a processor operatively coupled to one or more of the electrode and the microelectrodes, and while applying RF energy to the target tissue, the processor is capable of:
obtaining output signals from each of the bipolar microelectrode pairs, one or more of the output signals comprises an electrogram (EGM) reading; and
monitoring changes in frequency spectra of the EGMs in a frequency domain; and
an indicator indicating a level of lesion maturation that is proportional to changes in the frequency spectra of the EGM.

18. The system of claim 17, wherein the indicator is capable of indicating the level of lesion maturation during application of RF energy to the target tissue.

19. The system of claim 17, wherein the processor is capable of determining a level of one or more characteristics that are proportional to the frequency spectra, the one or more characteristics comprise contact force between the elongated member and the target tissue.

20. The system of claim 17, wherein the processor is capable of converting the EGM readings from a time domain to the frequency domain.
